(19) United States
(12) Patent Application Publication
(10) Pub. No.: US 2008/0172092 A1
(43) Pub. Date: Jul. 17, 2008

(54) SYSTEM AND METHOD FOR SPINAL INSTRUMENTATION

(76) Inventor: Paul Edward Kraemer, Seattle, WA (US)

Correspondence Address: WILEY REIN LLP, 1776 K. STREET N.W., WASHINGTON, DC, 20006, US

(21) Appl. No.: 11/972,025

(22) Filed: Jan. 10, 2008

Related U.S. Application Data
(60) Provisional application No. 60/880,066, filed on Jan. 12, 2007.
(60) Provisional application No. 60/929,758, filed on Jul. 11, 2007.

(51) Int. Cl. A61B 17/70; A61B 17/80; A61B 17/86

(52) U.S. Cl. 606/248; 606/70; 606/316; 606/279

(57) ABSTRACT

A spinal instrumentation system includes an oblong tension ring having a tension screw receptacle, a pair of compression balls having passages therethrough disposed within the tension ring, and a tension screw. Threading the tension screw into the tension screw receptacle inhibits movement of the compression balls relative to the tension ring and each other, thereby making the system rigid. Fixation screws are inserted through the passages in the compression balls and anchored in bone. One of the fixation screws may be a screw designed to traverse the lamina and including a scalloped segment. A pair of such systems may be posteriorly attached to the vertebrae, one on either side of the spinous process, to fuse the superior segment in a lumbar fusion procedure. The spinal fixation screws traverse the lamina, crossing at their scalloped segments.

### CROSS-REFERENCE TO RELATED APPLICATIONS

This application claims the benefit of United States provisional application no. 60/880,066, filed 12 Jan. 2007, and United States provisional application no. 60/929,758. The instrumenting are hereby incorporated by reference as though fully set forth herein.

### BACKGROUND OF THE INVENTION

a. Field of the Invention

The instant invention relates to generally to orthopedic surgery, and more specifically to spinal fusion surgery. More particularly, the instant invention relates to devices for and methods of instrumenting the superior segment in a lumbar fusion procedure.

b. Background Art

The spinal column is a complex system of bones and connective tissue that protects critical elements of the nervous system. Though complex, the spine is a highly flexible structure, capable of a high degree of curvature and twist through a wide range of motion.

It is known to attempt to correct spinal defects and restore stability to the spine through immobilization. Often, immobilization is accomplished through spinal fusion-the process of rigidly attaching multiple vertebrae, thereby reducing or eliminating freedom of movement in the fused segment of the spinal column. Spinal fusion typically employs spinal instrumentation, including screws, rods, and other connectors, which are anchored in vertebral bone on opposite sides of the segment of the spinal column to be fused.

Existing spinal instrumentation systems often present surgical difficulties when used in the superior (that is, the most cranial) segment of the region to be fused. Such difficulties include facet disease and adjacent segment disease, which may result from damage to the vascularity and innervation of the first cranial non-fused segment. In addition, spinal instrumentation procedures are often invasive, traumatic, and painful for the patient.

### BRIEF SUMMARY OF THE INVENTION

It is an object of the present invention to provide a spinal instrumentation system that preserves the vascularity and innervation of the first superjacent facet, thereby minimizing the likelihood of facet disease and adjacent segment disease.

Another object of the present invention is to provide a spinal instrumentation system suitable for use in minimally invasive posterior spinal fusion procedures.

Still another object of the present invention is to provide a spinal instrumentation system having reduced prominence.

A further object of the invention is to provide a spinal instrumentation system readily adaptable for multilevel fusions.

Yet another object of the present invention is to provide a spinal instrumentation system that reduces the possibility of pars fracture while achieving improved fixation in the lamina and posterior elements.

In a first aspect, the present invention provides a spinal instrumentation system for use in the superior segment of a lumbar fusion. The spinal instrumentation system generally includes: an oblong tension ring defining an opening and including a tension screw receptacle; a first compression ball having a first passage therethrough, the first passage being dimensioned to receive a first spinal fixation device; a second compression ball having a second passage therethrough, the second passage being dimensioned to receive a second spinal fixation device, the first compression ball and the second compression ball being configured to be disposed at least partially within the oblong tension ring; and a tension screw configured to be threaded into the tension screw receptacle, wherein threading the tension screw into the tension screw receptacle progressively inhibits movement of the first compression ball and the second compression ball relative to the tension ring and each other. Typically, the first compression ball and the second compression ball are configured to be substantially aligned with each other at least partially within a plane of the opening of the oblong tension ring. Preferably, an inner wall of the oblong tension ring is shaped to conform to an outer wall of the first compression ball and an outer wall of the second compression ball.

The system further includes: a first spinal fixation device configured to be inserted through the first passage; and a second spinal fixation device configured to be inserted through the second passage, wherein at least one of the first spinal fixation device and the second spinal fixation device is a pedicle screw. Preferably, at least one of the first spinal fixation device and the second spinal fixation device is a cortical bone screw comprising: a shank segment proximate a first end thereof; a threaded segment proximate a second end thereof; and a cutaway segment between the shank segment and the threaded segment, wherein the threaded segment has a circular axial cross-section and the cutaway segment has a non-circular axial cross-section having an area less than an area of the axial cross-section of the threaded segment.

In some embodiments of the invention, at least one of the first passage and the second passage is a keyhole-shaped passage having a central cylindrical portion dimensioned to receive the respective spinal fixation device and a slot portion adjacent the central cylindrical portion configured to permit expansion and reduction in an axial cross-section of the central cylindrical portion of the passage. Threading the tension screw into the tension screw receptacle may progressively reduce the axial cross-section of the central cylindrical portion of the keyhole-shaped passage.

It is also contemplated that the spacing between the first compression ball and the second compression ball may be increased through the use of an optional spacer dimensioned to be placed within the oblong tension ring and shaped to be disposed between the first compression ball and the second compression ball.

For use in multilevel fusions, the system may further include a connector rod coupled at a first end to the oblong tension ring and having a second end configured to be connected to a third spinal fixation device. The oblong tension ring optionally includes a slot, and the first end of the connector rod may include a slide portion disposed within the slot such that a position of the connector rod relative to the oblong tension ring is adjustable. Alternatively, the connector rod may be rigidly coupled to the oblong tension ring.

Typically, the tension screw will be wedge shaped, such as conical or frusto-conical (collectively referred to herein as “conical”), and the tension screw receptacle will be located at an end of the oblong tension ring. The tension screw receptacle may be oriented to receive the tension screw perpendicular to a plane of the opening defined by the oblong tension ring, or may be oriented to receive the tension screw parallel to a plane of the opening defined by the oblong tension ring.

Also disclosed herein is a spinal fixation screw, generally including: a shank segment proximate a first end thereof; a threaded segment proximate a second end thereof; and a cutaway segment between the shank segment and the threaded segment, wherein the threaded segment has a circular axial cross-section and the cutaway segment has a non-circular axial cross-section having an area less than an area of the axial cross-section of the threaded segment. The shank segment may also have a circular axial cross-section, which may be substantially congruent to the circular axial cross-section of the threaded segment.

Preferably, the threaded segment has a D-shaped axial cross-section including a substantially straight portion and an arcuate portion, where the arcuate portion has an arc length about two-thirds of a circumference of the circular axial cross-section of the threaded segment and is substantially aligned with a circumference of the circular axial cross-section of the threaded segment. A length of the cutaway segment will typically be between about one-third and about one-fourth of the overall length L1 of the spinal fixation screw.

Typically, the first end of the spinal fixation screw will include a tool receptacle configured to mate with a tool adapted to rotate the spinal fixation screw. It may also include an indicator that identifies a rotational orientation of the cutaway segment.

The present invention also provides a method of rigidly connecting articulated elements. The method generally includes the step of providing a fixation instrumentation system including: an oblong tension ring defining an opening and including a tension screw receptacle; a first compression ball having a first passage therethrough; a second compression ball having a second passage therethrough; and a tension screw configured for insertion into the tension screw receptacle. The first fixation device may be inserted through the first passage, and the second fixation device may be inserted through the second passage. The first and second compression balls may then be oriented such that the first and second fixation devices are oriented in respective first and second desired positions. By threading the tension screw into the tension screw receptacle, the first fixation device and the second fixation device may be secured, respectively, in the first desired position and the second desired position.

The method also typically includes: attaching the first fixation device to a first articulated element; and attaching the second fixation device to a second articulated element, thereby rigidly connecting the first articulated element to the second articulated element. For example, the first fixation device may be a cortical bone screw configured to obliquely traverse the lamina and pars interarticularis; the second fixation device may be a pedicle screw; the step of attaching the first fixation device to a first articulated element may include attaching the cortical bone screw to a cranial vertebral segment, traversing the lamina and ending in the pedicle of the cranial vertebral segment; and the step of attaching the second fixation device to a second articulated element may include attaching the pedicle screw to the pedicle of a caudal vertebral segment. The fixation instrumentation system is designed to be positioned posteriorly.

In another aspect, the invention provides a spinal instrumentation system for use in the superior segment of a lumbar fusion, including: a lamina plate having a cranial end and a caudal end and including at least one fixation hole therethrough; a first spinal fixation device dimensioned for insertion through the at least one fixation hole; a connector rod having a cranial end and a caudal end, the connector rod being movably coupled at its cranial end to the caudal end of the lamina plate via a multi-axial connector, wherein the caudal end of the connector rod is configured to be connected to a second spinal fixation device; and a locking structure positioned between the at least one fixation hole and the multi-axial connector, the locking structure being configured such that when the first spinal fixation device is introduced into the at least one fixation hole, the first spinal fixation device causes the locking structure to progressively inhibit movement of the connector rod, thereby locking the connector rod in position relative to the lamina plate. Preferably, the locking structure is further configured such that when the first spinal fixation device is introduced into the at least one fixation hole, the first spinal fixation device causes the locking structure to progressively inhibit movement of the first spinal fixation device, thereby locking the first spinal fixation device in position relative to the lamina plate. The locking structure is typically a compression locking structure, and the connector rod is typically coupled to the lamina plate via a ball joint.

In some embodiments of the invention, the first spinal fixation device is a cortical bone screw configured to obliquely traverse the lamina and pars interarticularis and the second spinal fixation device is a pedicle screw.

In addition, the at least one fixation hole may include a first fixation hole proximate the caudal end of the lamina plate and a second fixation hole proximate the cranial end of the lamina plate. Optionally, the at least one fixation hole may be internally threaded. A third spinal fixation device dimensioned for insertion through the second fixation hole, such as a cortical bone screw, may also be provided.

A length of the connector rod may be selected to span between a most cranial segment in the lumbar fusion and a most caudal segment in the lumbar fusion, thereby making the system adaptable for use in a multilevel fusion.

An advantage of the present invention is that it preserves vascularity and innervation of the first superjacent facet, minimizing the likelihood of facet disease and adjacent segment disease.

Another advantage of the present invention is that it is usable with limited surgical exposure.

A further advantage of the present invention is that it has reduced prominence, thereby reducing patient pain and enhancing the usability of the spinal instrumentation system at the thoracolumbar junction and in thinner patients.

Yet another advantage of the present invention is that it is suitable for use in multilevel fusions.

Still another advantage of the present invention is that it can be rigidly locked simply and efficiently.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a top schematic view of a spinal instrumentation system according to a first embodiment of the invention.

FIG. 2 is a side view of a spinal instrumentation system according to a first embodiment of the invention.

FIGS. 3A and 3B illustrate the insertion and orientation of spinal fixation devices into the spinal instrumentation system.

FIG. 4 is a cross-section of a spinal instrumentation system taken along line 4-4 in FIG. 2.

FIG. 5 illustrates a spinal instrumentation system including a spacer.

FIG. 6A illustrates a spinal instrumentation system according to a second embodiment of the invention, usable to good advantage in multilevel fusions.

FIG. 6B is a detail of region 6B in FIG. 6A.

FIG. 7 illustrates a spinal fixation device according to an embodiment of the present invention.

FIG. 8 is a cross-section taken along line 8-8 in FIG. 7.

FIG. 9 is a cross-section taken along line 9-9 in FIG. 7.

FIG. 10 is a cross-section taken along line 10-10 in FIG. 7.

FIG. 11 illustrates a single-level lumbar fusion using a spinal instrumentation system according to an embodiment of the present invention viewed posteriorly.

FIG. 12 illustrates a single-level lumbar fusion using a spinal instrumentation system according to an embodiment of the present invention viewed laterally.

FIG. 13 illustrates a single-level lumbar fusion using a spinal instrumentation system according to an embodiment of the present invention viewed along the spinal column.

FIG. 14 depicts a spinal instrumentation system according to another embodiment of the invention.

FIGS. 15A through 15D are cross-sections taken along line 15-15 in FIG. 14 that schematically illustrate the sequence of locking the spinal instrumentation system shown in FIG. 14.

FIGS. 16A through 16C are dorsal projections schematically illustrating the sequence of locking the spinal instrumentation system depicted in FIG. 14.

FIG. 17 illustrates a single-level lumbar fusion using the spinal instrumentation system of FIG. 14 viewed posteriorly.

FIG. 18 illustrates a single-level lumbar fusion using the spinal instrumentation system of FIG. 14 viewed laterally.

FIG. 19 illustrates a single-level lumbar fusion using the spinal instrumentation system of FIG. 14 viewed along the spinal column.

### DETAILED DESCRIPTION OF THE INVENTION

The present invention provides devices and methods for rigidly connecting articulated elements. For the sake of explanation, the present invention will be described in connection with orthopedic or neurosurgical spinal surgery, more particularly in the context of spinal instrumentation, and most particularly with reference to instrumentation of the superior (that is, most cranial) segment in a lumbar fusion procedure. One of ordinary skill in the art will appreciate, however, that the teachings disclosed herein may be applied to good advantage in other contexts where it is desirable to rigidly connect articulated elements.

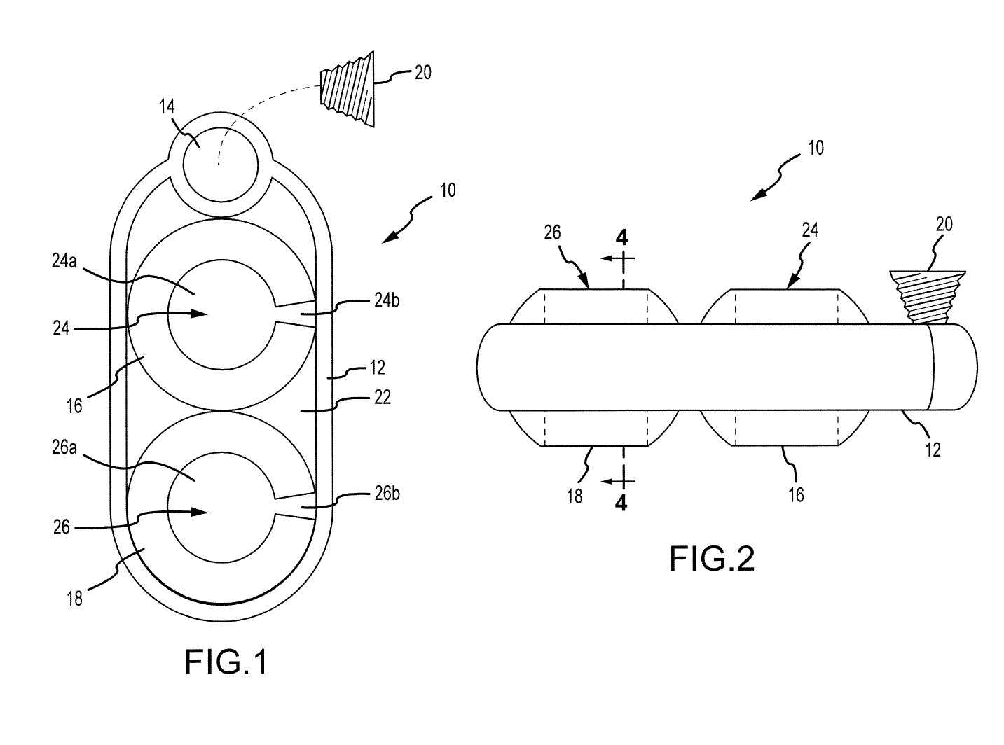

FIG. 1 schematically illustrates a spinal instrumentation system 10 according to one embodiment of the present invention. Spinal instrumentation system 10 generally includes a tension ring 12 including a tensioning device receptacle 14, a first compression ball 16, and a second compression ball 18. Spinal instrumentation system 10 also includes a tensioning device configured to be inserted into tensioning device receptacle 14. For example, a tension screw 20 may be configured to be threaded into tensioning device receptacle 14. Suitable materials for tension ring 12 and compression balls 16, 18 include titanium and other biocompatible materials.

Tension ring 12 is preferably an oblong ring. The term “oblong” is used herein to refer to any elongate shape, including, but not limited to, elliptical rings, oval (e.g., racetrack-shaped) rings, and the like. Tension ring 12 defines an opening 22 within which first compression ball 16 and second compression ball 18 are at least partially disposed. As shown in FIG. 2, first compression ball 16 and second compression ball 18 are substantially aligned with each other at least partially within the plane of opening 22 defined by tension ring 12. By “substantially aligned,” it is meant that first compression ball 16 and second compression ball 18 are not staggered relative to the plane of opening 22 in tension ring 12.

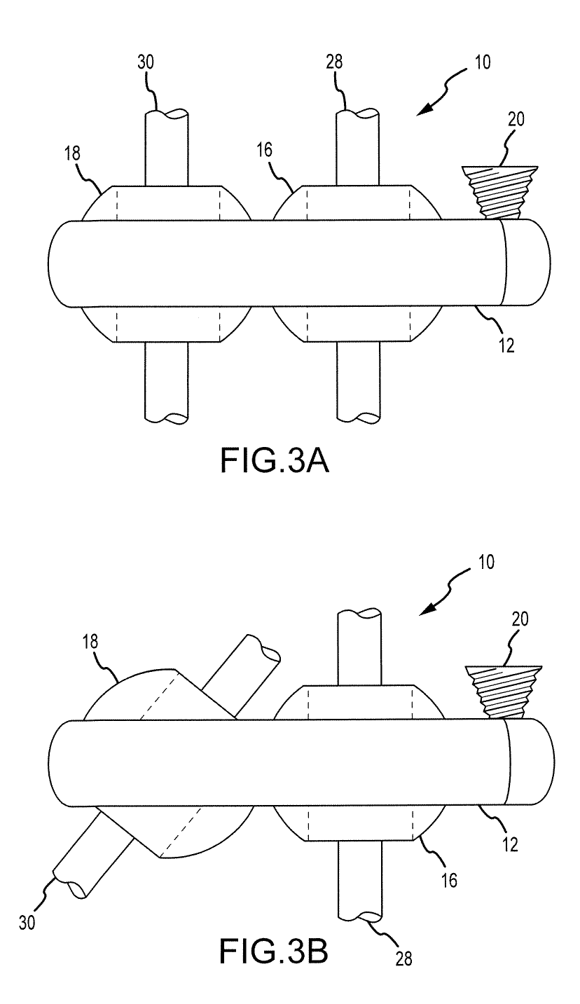

As shown in FIGS. 1 and 2, first compression ball 16 and second compression ball 18 include respective first and second passages 24, 26 therethrough. First passage 24 and second passage 26 are dimensioned to receive a first spinal fixation device 28 and a second spinal fixation device 30, respectively. First and second spinal fixation devices 28, 30 are shown received through first and second passages 24, 26 in FIGS. 3A and 3B. Typically, at least one of first and second spinal fixation devices 28, 30 will be a pedicle screw as generally known in the art.

As illustrated in FIG. 1, first and second passages 24, 26 may be keyhole-shaped passages including central cylindrical portions 24a, 26a and adjacent slot portions 24b, 26b. Central cylindrical portions 24a, 26a receive spinal fixation devices 28, 30, while the adjacent slot portions 24b, 26b permit expansion and reduction in the size (that is, the axial cross-section) of cylindrical portions 24a, 26a. By reducing the size of central cylindrical portions 24a, 26a, fixation devices 28, 30 passing therethrough may be secured when spinal instrumentation system 10 is locked (described in further detail below).

As described above, first and second compression balls 16, 18 are held within tension ring 12. When spinal instrumentation system 10 is unlocked, first and second compression balls 16, 18 can rotate relative to each other as shown in FIGS. 3A and 3B. It is contemplated that compression balls 16, 18 may be able to rotate up to and including about 360 degrees about substantially any axis when spinal instrumentation system 10 is unlocked. This allows passages 24, 26, and therefore spinal fixation devices 28, 30 passing therethrough, to be oriented as desired prior to rigidly locking spinal instrumentation system 10.

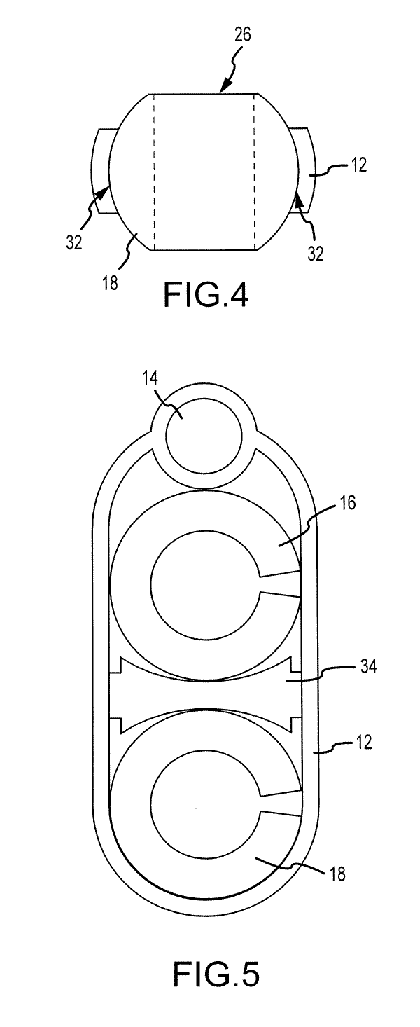

As shown in FIG. 4, it is desirable for an inner wall 32 of tension ring 12 to be shaped to substantially conform to an outer wall (that is, an outer profile or outer shape) of first and second compression balls 16, 18. This facilitates positive retention of compression balls 16, 18 within tension ring 12. In addition, it increases the contact surface area between compression balls 16, 18 and tension ring 12, increasing friction therebetween and advantageously enhancing the rigidity of spinal instrumentation system 10 when locked.

Similarly, in some embodiments of the invention, first and second compression balls 16, 18 are adjacent one another in order to promote frictional engagement between compression balls 16, 18 and enhance the rigidity of spinal instrumentation system 10 when locked. It is contemplated, however, that the spacing between compression balls 16, 18 may be adjustable through the use of a spacer 34, as illustrated in FIG. 5, to customize spinal instrumentation system 10 for a particular application (e.g., a particular patient's anatomy). Where spacer 34 is employed, compression balls 16, 18 will frictionally engage spacer 34, rather than each other, when spinal instrumentation system 10 is locked. Of course, it is within the spirit and scope of the invention to use different size spacers and/or different numbers of spacers to adjust and customize the spacing between compression balls 16, 18 for a particular application of spinal instrumentation system 10.

The tensioning device (e.g., tension screw 20) is operable to lock spinal instrumentation system 10. In some embodiments of the invention, tensioning device receptacle 14 is positioned at an end of tension ring 12 and is oriented to receive the tensioning device perpendicular to the plane of opening 22 in tension ring 12. Typically, the tensioning device will be wedge-shaped, such as the illustrated conical shape (a term used herein to encompass not only wholly conical shapes, but also frusto-conical shapes) of tension screw 20. One of ordinary skill in the art will appreciate, however, that other configurations and orientations of the tensioning device and tensioning device receptacle 14 may be employed without departing from the spirit and scope of the present invention. For example, it is contemplated that tensioning device receptacle 14 may be oriented to receive the tensioning device parallel to the plane of opening 22 in tension ring 12.

Spinal instrumentation system is locked by driving tension screw 20 into tensioning device receptacle 14. As conical tension screw 20 is driven (e.g., threaded) into tensioning device receptacle 14, tension ring 12 is placed into tension and compression balls 16, 18 are compressed. Movement of first and second compression balls 16, 18 relative to each other and relative to tension ring 12 is thereby progressively inhibited.

When spinal instrumentation system 10 is fully locked, it will be substantially rigid-that is, little or no relative movement will be possible between compression balls 16, 18 and tension ring 12. In addition, as compression balls 16, 18 are compressed, the axial cross-sectional area of central cylindrical portions 24a, 26a will be progressively reduced, thereby securing fixation devices 28, 30 within passages 24, 26. Further, compression balls 16, 18 will be pushed closer together as tension screw 20 is received in tensioning device receptacle 14, which adds desirable lordosis to spinal instrumentation system 10.

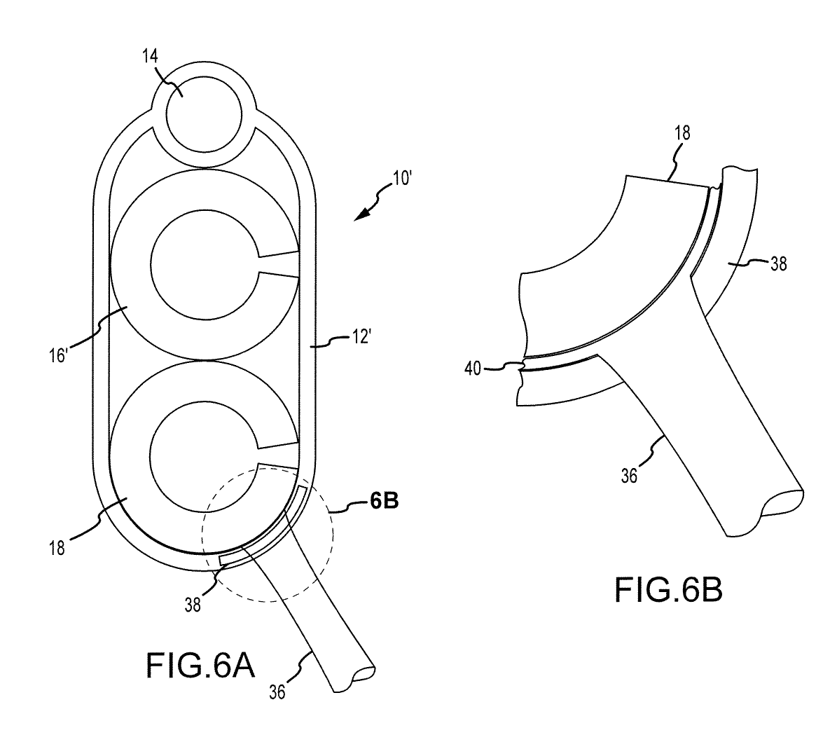

Another embodiment of a spinal instrumentation system according to the present invention, denoted 10′, is illustrated in FIG. 6A. Spinal instrumentation system 10′ may be employed to good advantage in multilevel lumbar fusions. Spinal instrumentation system 10′ further includes a connector rod 36 having a first end and a second end. The first end of connector rod 36 is coupled to tension ring 12′, while the second end of connector rod 36 is configured to be connected to a third spinal fixation device, such as a pedicle screw embedded in a most caudal vertebra in the fusion (not shown).

The length, orientation, and other geometry of connector rod 36 may be adjusted without departing from the spirit and scope of the present invention. For example, connector rod 36 may extend from tension ring 12′ as shown in FIG. 6A, or may extend from tension ring 12′ in mirror image fashion. Preferably, however, connector rod 36 is angled in both medial lateral and lordosis.

In some embodiments of spinal instrumentation system 10′, tension ring 12′ includes a slot 38, and the first end of connector rod 36 includes a slide portion 40 (FIG. 6B) disposed within slot 38. This permits a position of connector rod 36 to be adjusted relative to tension ring 12′. Preferably, as shown in the detail of FIG. 6B, slide portion 40 of connector rod 36 is positioned so as to be constrained when the tensioning device is received within tensioning device receptacle 14. Of course, it is also contemplated that connector rod 36 may be rigidly attached to tension ring 12′.

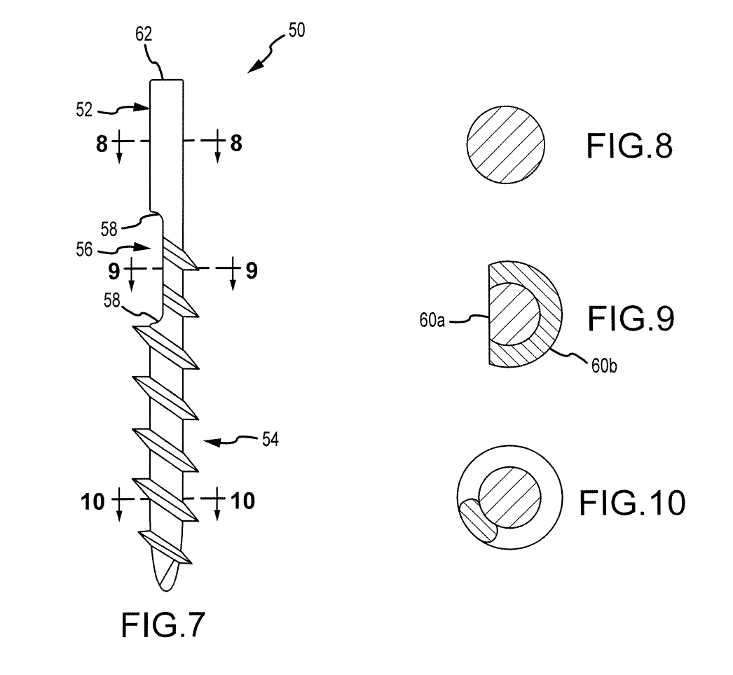

FIGS. 7-10 illustrate a spinal fixation screw that may be used to good advantage in conjunction with spinal instrumentation systems according to the present invention. The spinal fixation screw 50 illustrated in FIG. 7 is a cortical bone screw that is advantageously configured to obliquely traverse the lamina and pars interarticularis.

Spinal fixation screw 50 generally includes a shank segment 52 proximate a first end, a threaded segment 54 proximate a second end, and a cutaway (or “scalloped”) segment 56 between shank segment 52 and threaded segment 54. As illustrated in FIGS. 9 and 10, threaded segment 54 has a circular axial cross-section, while cutaway segment 56 has a non-circular axial cross-section. The axial cross-sectional area of cutaway segment 56 is less than the axial cross-sectional area of threaded segment 54. Shank segment 52 may also have a circular axial cross-section, as shown in FIG. 8, which may be substantially congruent to the axial cross-section of threaded segment 54. Where the axial cross-section of spinal fixation screw 50 changes, it is desirable to have a smooth transition between segments (as shown in FIG. 7 at transition points 58) in order to avoid stress concentration within spinal fixation screw 50.

Typically, the terminal end of spinal fixation screw 50 will not be self-tapping. This advantageously enhances the holding power of spinal fixation screw 50 in bone. It is within the spirit and scope of the invention, however, for spinal fixation screw 50 to be self-tapping.

Preferably, cutaway segment 56 has a D-shaped axial cross-section including a substantially straight portion 60a and an arcuate portion 60b as shown in FIG. 9. Arcuate portion 60b will typically have an arc length about two-thirds of a circumference of threaded segment 54, and will typically be substantially aligned with the circumference of threaded segment 54 in order to avoid stress concentration within spinal fixation screw 50. In addition, the length of cutaway segment 56 will typically be between about one-third and about one-fourth of the total length of spinal fixation screw 50. One of ordinary skill in the art will appreciate that the dimensions (e.g., overall length, diameter, thread pitch, and the like) of spinal fixation screw 50 may vary with particular applications, but it is contemplated that a suitable diameter of threaded segment 54 may be about 4.5 mm.

Advantageously, the reduced axial cross-section of cutaway segment 56 permits two spinal fixation screws 50 to cross each other with minimal clearance and reduced overall thickness, as will be described in further detail below.

To assist in identifying the orientation of cutaway segment 56 (e.g., the direction in which straight portion 60a is facing), the head 62 of spinal fixation screw 50 may include an indicator, such as an arrow, that identifies a rotational orientation of cutaway segment 56. Head 62 will also typically include a receptacle configured to mate with a tool adapted to rotate the spinal fixation screw. Of course, the receptacle and the indicator may be one and the same. In other embodiments, head 62 may be shaped to mate with a tool adapted to rotate the spinal fixation screw (e.g., shaped to mate with a hex head screwdriver). Still other configurations are contemplated (including, for example, shaping all or part of head 62 conically such that it progressively impinges on an adjacent structure when in use).

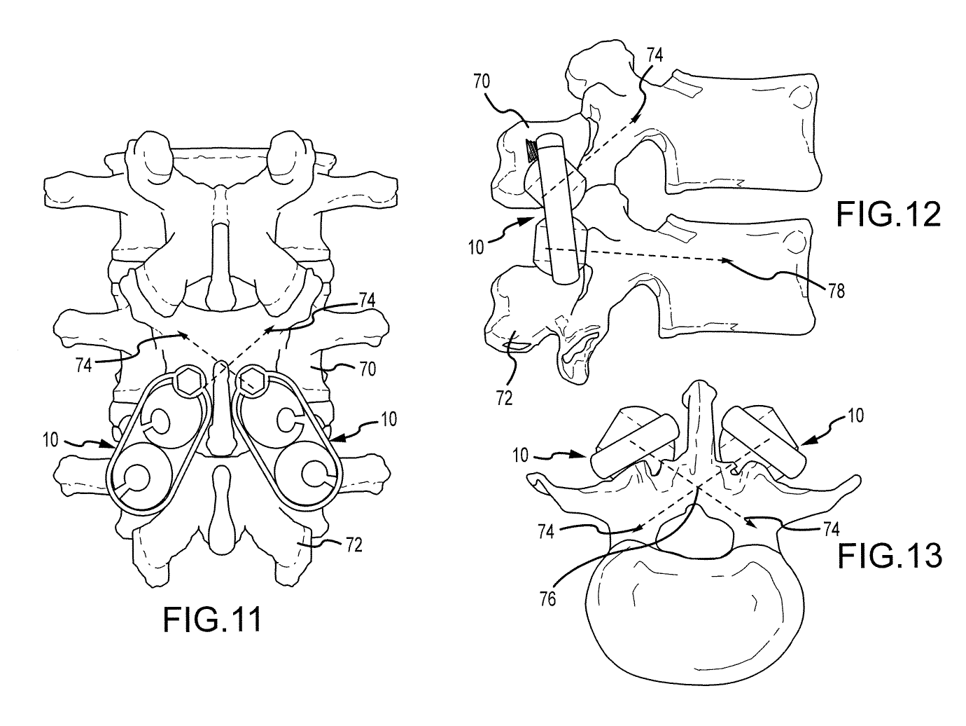

Use of spinal instrumentation system 10 to fuse a first articulated element (e.g., cranial vertebra 70) and a second articulated element (e.g., caudal vertebra 72) will be described with reference to FIGS. 11 through 13. As shown in FIGS. 11 through 13, two spinal instrumentation systems 10 are applied posteriorly, one on either side of the spinous process, in the most cranial segment of a lumbar fusion surgery. Application of both spinal instrumentation systems 10 generally follows the same steps.

First fixation device 28, preferably spinal fixation screw 50, is inserted through first compression ball 16 to be oriented cranially, and second fixation device 30, typically a pedicle screw, is inserted through second compression ball 18 to be oriented caudally. With spinal instrumentation system 10 unlocked, first and second compression balls 18, 20 may oriented such that their respective fixation devices 28, 30 are positioned as desired for insertion into bone.

As shown schematically in FIG. 13 (arrows 74), first fixation device 28 (e.g., spinal fixation screw 50) is anchored in cranial vertebra 70, beginning on the contralateral lamina, just across the midline and at the level of the lateral pars, traversing the lamina and ending in the pedicle. First fixation devices 28 cross at crossing zone 76. Crossing zone 76 preferably coincides with cutaway segments 56 such that first fixation devices 28 overlap at the reduced axial cross-section (e.g., straight portion 60a against straight portion 60a). In addition to reducing the overall depth of instrumentation within crossing zone 76, positioning fixation devices 28 flat-against-flat (e.g., reduced cross-section against reduced cross-section) reduces the likelihood of loosening of fixation devices 28 by backout.

As shown schematically in FIG. 12 (arrow 78), second fixation device 30 (e.g., a pedicle screw) is anchored in caudal vertebra 72 as generally known in the art. One of ordinary skill will also appreciate how to extend the principles disclosed herein to multilevel fusions, including the use of connector rods 26.

Spinal instrumentation system 10 can be made rigid by driving tension screw 20 or other suitable tensioning device into tensioning device receptacle 14, thereby locking spinal instrumentation system 10 as described above and completing the fusion between cranial vertebra 70 and caudal vertebra 72 in the superior segment of the lumbar fusion. The remainder of the lumbar fusion (e.g., the caudal levels) can be accomplished using pedicle screw instrumentation as generally known in the art.

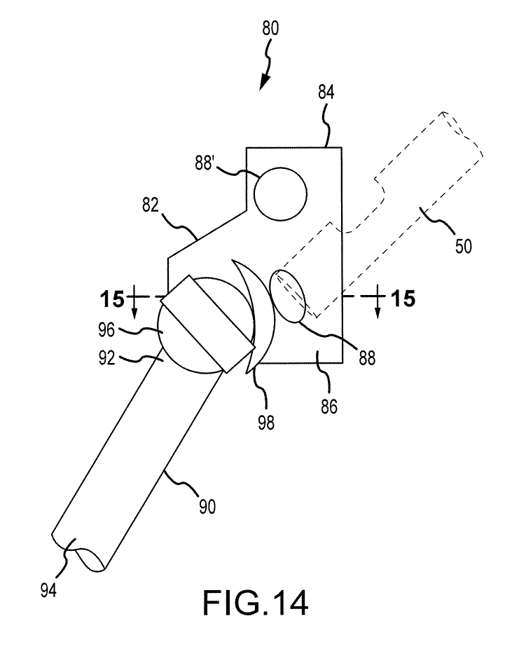

Another embodiment of a spinal instrumentation system according to the present invention, denoted 80, is illustrated in FIG. 14. Spinal instrumentation system 80 includes a lamina plate 82 (that is, a plate that generally conforms to the anatomy of the lamina) having a cranial end 84, a caudal end 86, and at least one fixation hole 88 therethrough. A connector rod 90 having a cranial end 92 and a caudal end 94 is movably coupled at cranial end 92 to caudal end 86 of lamina plate 82 via a multi-axial connector 96, such as a ball joint. (The term “ball joint” as used herein refers to any type of ball joint, whether the ball is a full ball or only a partial ball.) The length of connector rod 90 is selected to span between a most cranial segment in the lumbar fusion and a most caudal segment in the lumbar fusion. When spinal instrumentation system 80 is unlocked, connector rod 90 is freely movable relative to lamina plate 82 (that is, the orientation of connector rod 90 relative to lamina plate 82 may be adjusted).

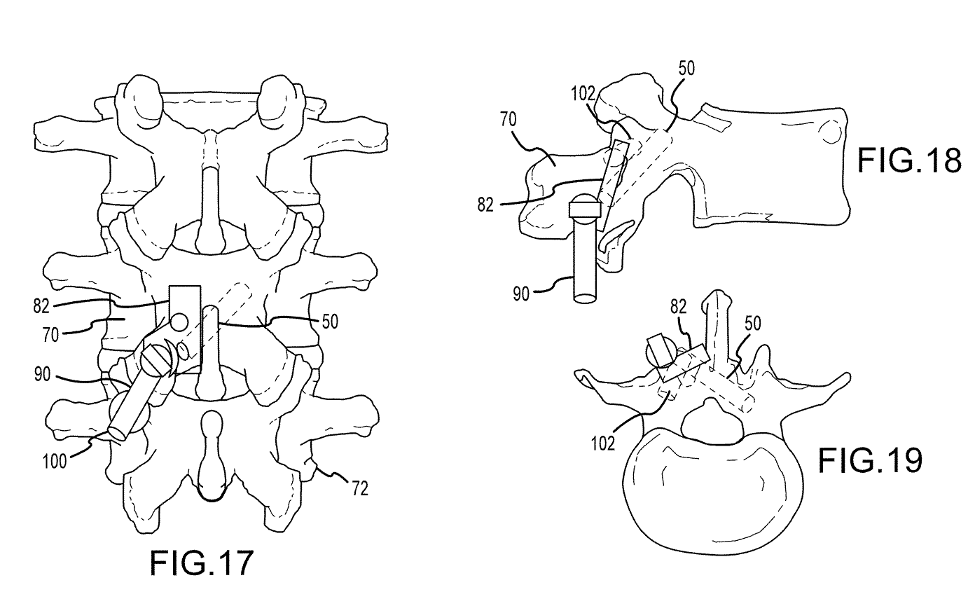

A first spinal fixation device, preferably spinal fixation screw 50 (described above and shown schematically in phantom in FIG. 14), is dimensioned for insertion through fixation hole 88. Once so inserted, spinal fixation screw 50 will be anchored in the vertebra, beginning on the contralateral lamina, just across the midline and at the level of the lateral pars, traversing the lamina and ending in the pedicle. Caudal end 94 of connector rod 90 is configured to be connected to a second spinal fixation device, such as a pedicle screw (not shown in FIG. 14, but illustrated schematically in FIG. 17).

A locking structure 98, which is typically a hollow compression locking structure, is positioned between multi-axial connector 96 and fixation hole 88. Locking structure 98 is configured such that, when spinal fixation screw 50 is introduced into fixation hole 88, spinal fixation screw 50 bears upon and deforms locking structure 98, which in turn bears upon multi-axial connector 96, thereby progressively inhibiting movement of connector rod 90, and eventually locking connector rod 90 in position relative to lamina plate 82. Similarly, as spinal fixation screw 50 is introduced into fixation hole 88, the restorative forces arising in locking structure 98 will cause locking structure 98 to bear upon spinal fixation screw 50, progressively inhibiting movement thereof and eventually locking spinal fixation screw 50 in position relative to lamina plate 82.

Thus, to lock spinal instrumentation system 80, one need only insert spinal fixation screw 50 (or other suitable fixation device) into fixation hole 88, which deforms locking structure 98 and wedges locking structure 98 between multi-axial connector 96 and spinal fixation screw 50. This creates a “fixed-angle” interference fit (or “cold weld”) between connector rod 90 and spinal fixation screw 50 that is substantially rigid in substantially all planes.

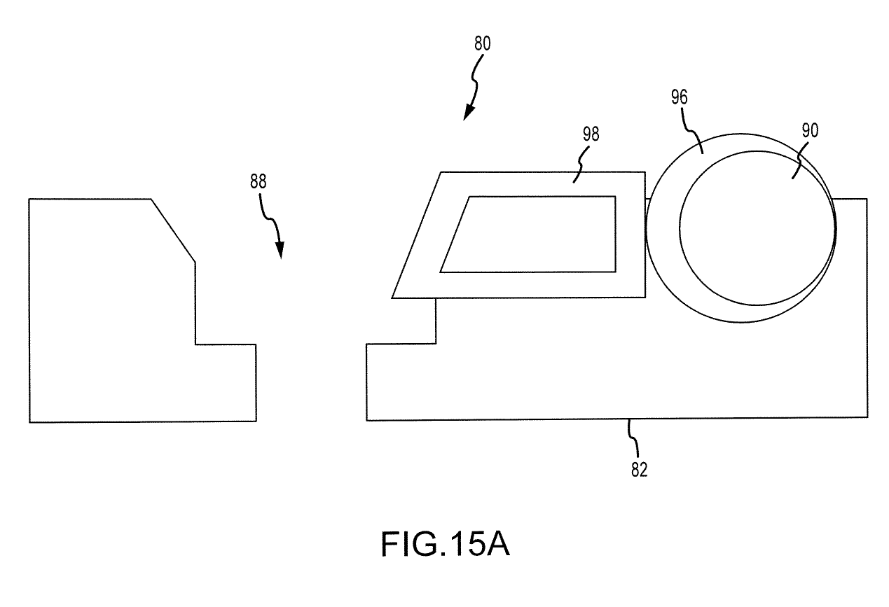

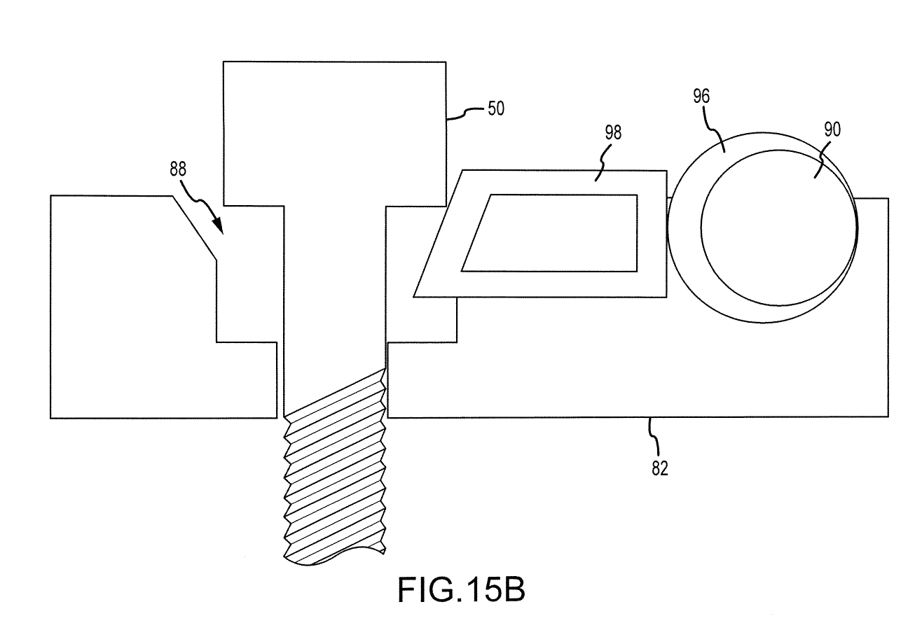

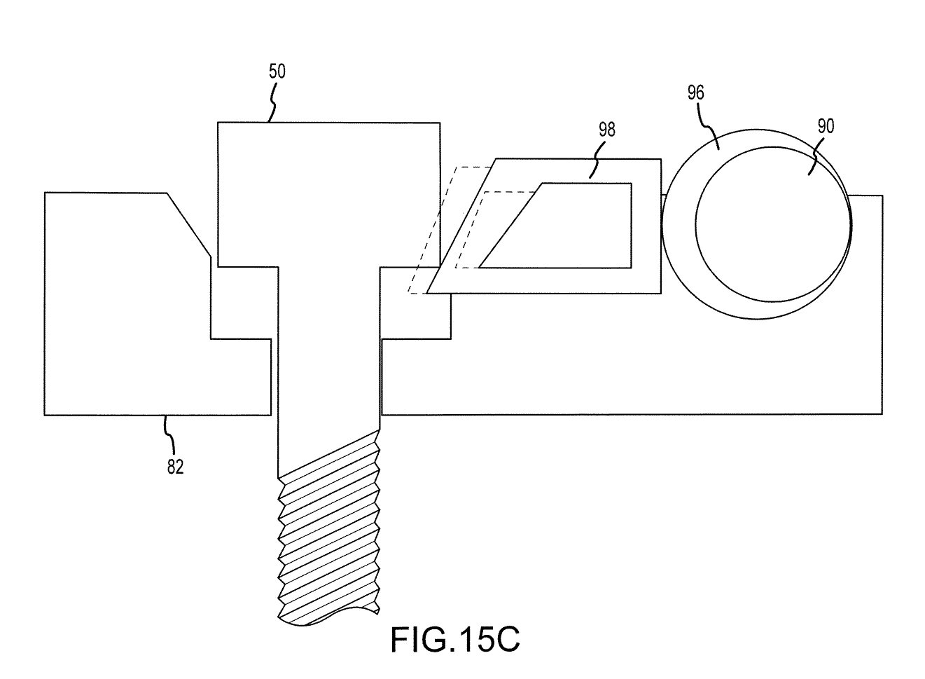

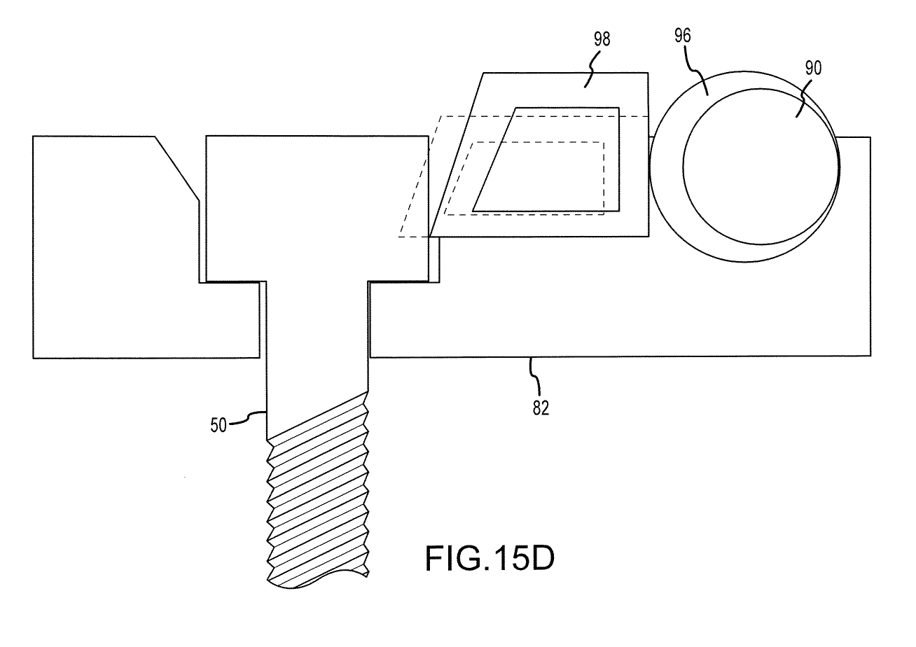

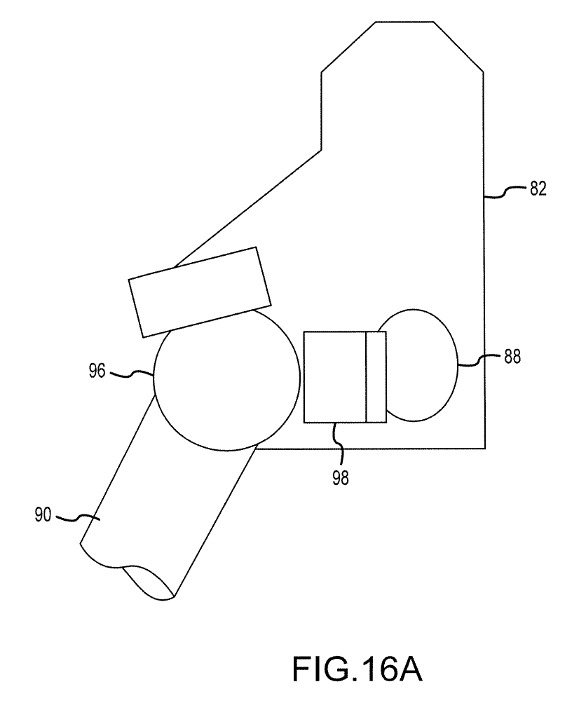

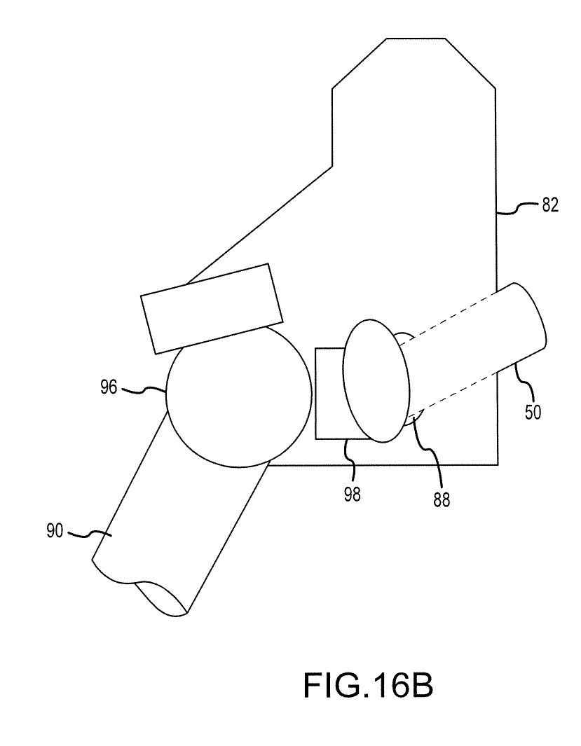

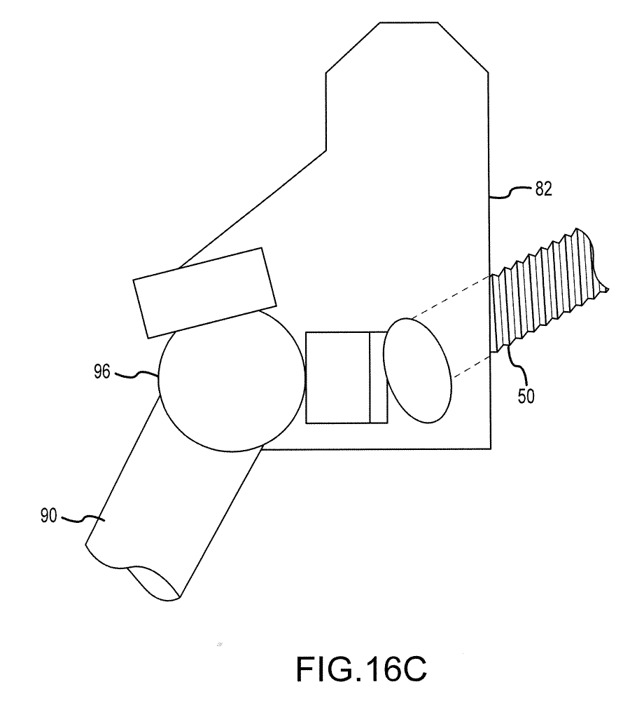

Advantageously, the angles of connector rod 90 and spinal fixation screw 50, both relative to one another and relative to lamina plate 82, are not substantially rigidly fixed until spinal fixation screw 50 is fully inserted through fixation hole 88. This is illustrated in FIGS. 15A-15D and 16A-16C. FIGS. 15A and 16A illustrate spinal instrumentation system 80 unlocked, without spinal fixation screw 50 inserted into fixation hole 88. In FIGS. 15B and 16B, spinal fixation screw 50 has been partially inserted into fixation hole 88, with the head of spinal fixation screw 50 just in contact with locking structure 98.

In FIG. 15C, spinal fixation screw 50 has been threaded further into fixation hole 88 such that the head of spinal fixation screw 50 has partially compressed locking structure 98 (the undeformed configuration of locking structure 98 is shown in phantom). Thus, FIG. 15C shows spinal instrumentation system 80 in a partially locked state-locking structure 98 is bearing on both multi-axial connector 96 and spinal fixation screw 50, but connector rod 90 and spinal fixation screw 50 are not yet substantially rigidly locked.

FIGS. 15D and 16C illustrate spinal instrumentation system 80 in a fully locked state, with spinal fixation screw 50 fully inserted. In FIG. 15D, the undeformed configuration of locking structure 98 is shown in phantom. The interference fit between the head of spinal fixation screw 50, fully compressed locking device 98, and multi-axial connector 96 renders spinal instrumentation system 80 substantially rigid in substantially all planes with connector rod 90 and spinal fixation screw 50 held relative to each other in a substantially fixed angle.

To enhance attachment of lamina plate 82 to bone, lamina plate 82 may include multiple fixation holes, such as a first fixation hole 88 proximate caudal end 86 and a second fixation hole 88′ proximate cranial end 84. A third spinal fixation device, such as a pedicle screw (not shown in FIG. 14, but illustrated schematically in FIGS. 18 and 19), may be provided for insertion through second fixation hole 88′. It is also contemplated that first fixation hole 88 and/or second fixation hole 88′ may be internally threaded to further positively restrain the spinal fixation screws passing therethrough.

Performance of a single-level lumbar fusion using spinal instrumentation system 80 will be described with reference to FIGS. 17-19. Though FIGS. 17-19 depict only a single spinal instrumentation system 80, one of ordinary skill will recognize that two spinal instrumentation systems 80, mounted posteriorly and on opposing sides of the spinous process, would typically be employed, with their respective first spinal fixation devices crossing as described above.

Lamina plate 82 is attached to cranial vertebra 70 via a first spinal fixation device, such as spinal fixation screw 50 (shown in phantom), inserted through fixation hole 88. Connector rod 90 extends caudally towards caudal vertebra 72, where it is attached to a second spinal fixation device, such as pedicle screw 100 (shown schematically) anchored in caudal vertebra 72. If desired, a third spinal fixation device, such as an additional pedicle screw 102 (shown in phantom) may be inserted through fixation hole 88′ to further anchor lamina plate 82 to cranial vertebra 70.

As described above, spinal instrumentation system 80 becomes rigidly locked via insertion of spinal fixation screw 50, or other suitable spinal fixation device, through fixation hole 88. The remainder of the lumbar fusion (e.g., the caudal levels) can be accomplished using pedicle screw instrumentation as generally known in the art.

Spinal instrumentation system 80 advantageously provides enhanced fixation in the lamina and posterior elements via multiple planes of screw fixation. In addition, spinal instrumentation system 80 provides a lower profile and reduced prominence of instrumentation. Further, because a length of connector rod 90 can be selected to span between cranial and caudal segments in a lumbar fusion, spinal instrumentation system 80 lends itself well to multilevel lumbar fusions.

Advantageously, the devices and methods disclosed herein are minimally invasive and spare the vascularity and innervation of the first superjacent facet (that is, the first cranial non-fused segment). Therefore, the devices and methods disclosed herein reduce the risk of facet disease and coexistent junctional (adjacent facet) disease.

Although several embodiments of this invention have been described above with a certain degree of particularity, those skilled in the art could make numerous alterations to the disclosed embodiments without departing from the spirit or scope of this invention. For example, additional fixation holes may be provided in lamina plate 82 as desired to anchor lamina plate 82 to bone.

Similarly, any suitable multi-axial connector may be used to connect connector rod 90 to lamina plate 82, including, but not limited to, the ball joint described above. As another example, though lamina plate has been described above as generally conforming to the size and shape of a hemi-lamina, it is contemplated that it may also have an area substantially smaller than that of the hemi-lamina.

Further, though the invention has been described including the use of spinal fixation screw 50, other fixation devices may be used without departing from the spirit and scope of the present invention. Likewise, one of ordinary skill in the art will appreciate that the type of head of spinal fixation screw 50 may be independent of the type of shaft of spinal fixation screw 50. For example, when used in conjunction with spinal instrumentation system 80, the head of spinal fixation screw 50 may have a diameter larger than the space between locking structure 98 and fixation hole 88 when spinal instrumentation system 80 is unlocked, and about equal to the space between locking structure 98 and fixation hole 88 when spinal instrumentation system 80 is locked (see FIGS. 15A-15D).

Moreover, though the invention has been described as including a pair of compression balls, more or fewer compression balls may be used if so desired. Likewise, it should be understood that the compression balls need not be perfectly spherical in shape. It is expressly contemplated that the compression balls may be frusto-spherical or even disk-like without departing from the scope of the invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A spinal instrumentation system for use in the superior segment of a lumbar fusion, the system comprising:
an oblong tension ring defining an opening and including a tension screw receptacle;
a first compression ball having a first passage therethrough, the first passage being dimensioned to receive a first spinal fixation device;
a second compression ball having a second passage therethrough, the second passage being dimensioned to receive a second spinal fixation device,
the first compression ball and the second compression ball being configured to be disposed at least partially within the oblong tension ring; and
a tension screw configured to be threaded into the tension screw receptacle,
wherein threading the tension screw into the tension screw receptacle progressively inhibits movement of the first compression ball and the second compression ball relative to the tension ring and each other.

2. The system according to claim 1, wherein the first compression ball and the second compression ball are configured to be substantially aligned with each other at least partially within a plane of the opening of the oblong tension ring.

3. The system according to claim 1, further comprising:
a first spinal fixation device configured to be inserted through the first passage; and
a second spinal fixation device configured to be inserted through the second passage,
wherein at least one of the first spinal fixation device and the second spinal fixation device is a pedicle screw.

4. The system according to claim 1, wherein at least one of the first spinal fixation device and the second spinal fixation device is a cortical bone screw comprising:
a shank segment proximate a first end thereof;
a threaded segment proximate a second end thereof; and
a cutaway segment between the shank segment and the threaded segment,
wherein the threaded segment has a circular axial cross-section and the cutaway segment has a non-circular axial cross-section having an area less than an area of the axial cross-section of the threaded segment.

5. The system according to claim 4, wherein the threaded segment has a D-shaped axial cross-section including a substantially straight portion and an arcuate portion.

6. The system according to claim 5, wherein the arcuate portion has an arc length about two-thirds of a circumference of the circular axial cross-section of the threaded segment.

7. The system according to claim 5, wherein the arcuate portion is substantially aligned with a circumference of the circular axial cross-section of the threaded segment.

8. The system according to claim 4, wherein the cortical bone screw has a length L1, and a length of the cutaway segment is between about one-third and about one-fourth of L1.

9. The system according to claim 1, wherein at least one of the first passage and the second passage is a keyhole-shaped passage having a central cylindrical portion dimensioned to receive the respective spinal fixation device and a slot portion adjacent the central cylindrical portion configured to permit expansion and reduction in an axial cross-section of the central cylindrical portion of the passage.

10. The system according to claim 9, wherein threading the tension screw into the tension screw receptacle progressively reduces the axial cross-section of the central cylindrical portion of the keyhole-shaped passage.

11. The system according to claim 1, further comprising a spacer dimensioned to be placed within the oblong tension ring and shaped to be disposed between the first compression ball and the second compression ball, thereby to increase a separation between the first compression ball and the second compression ball.

12. The system according to claim 1, further comprising a connector rod coupled at a first end to the oblong tension ring and having a second end configured to be connected to a third spinal fixation device.

13. The system according to claim 12, wherein the oblong tension ring includes a slot, and wherein the first end of the connector rod comprises a slide portion disposed within the slot such that a position of the connector rod relative to the oblong tension ring is adjustable.

14. The system according to claim 12, wherein the connector rod is rigidly coupled to the oblong tension ring.

15. The system according to claim 1, wherein an inner wall of the oblong tension ring is shaped to conform to an outer wall of the first compression ball and an outer wall of the second compression ball.

16. The system according to claim 1, wherein the tension screw is conical.

17. The system according to claim 1, wherein the tension screw receptacle is oriented to permit receipt of the tension screw perpendicular to a plane of the opening defined by the oblong tension ring.

18. The system according to claim 1, wherein the tension screw receptacle is oriented to permit receipt of the tension screw parallel to a plane of the opening defined by the oblong tension ring.

19. The system according to claim 1, wherein the tension screw receptacle is located at an end of the oblong tension ring.

20. A spinal fixation screw comprising:
a shank segment proximate a first end thereof;
a threaded segment proximate a second end thereof, and
a cutaway segment between the shank segment and the threaded segment,
wherein the threaded segment has a circular axial cross-section and the cutaway segment has a non-circular axial cross-section having an area less than an area of the axial cross-section of the threaded segment.

21. The screw according to claim 20, wherein the threaded segment has a D-shaped axial cross-section including a substantially straight portion and an arcuate portion.

22. The screw according to claim 21, wherein the arcuate portion has an arc length about two-thirds of a circumference of the circular axial cross-section of the threaded segment.

23. The screw according to claim 21, wherein the arcuate portion is substantially aligned with a circumference of the circular axial cross-section of the threaded segment.

24. The screw according to claim 20, wherein the spinal fixation screw has a length L1, and a length of the cutaway segment is between about one-third and about one-fourth of L1.

25. The screw according to claim 20, wherein the shank segment has a circular axial cross-section substantially congruent to the circular axial cross-section of the threaded segment.

26. The screw according to claim 20, wherein the first end of the spinal fixation screw is configured to mate with a tool adapted to rotate the spinal fixation screw.

27. The screw according to claim 20, wherein the first end of the spinal fixation screw includes an indicator that identifies a rotational orientation of the cutaway segment.

28. A method of rigidly connecting articulated elements, the method comprising:
providing a fixation instrumentation system, the fixation instrumentation system comprising:
an oblong tension ring defining an opening and including a tension screw receptacle;
a first compression ball having a first passage therethrough;
a second compression ball having a second passage therethrough; and
a tension screw configured for insertion into the tension screw receptacle;
inserting a first fixation device through the first passage;
inserting a second fixation device through the second passage;
orienting the first compression ball such that the first fixation device is oriented in a first desired position;
orienting the second compression ball such that the second fixation device is oriented in a second desired position; and
threading the tension screw into the tension screw receptacle, thereby securing the first fixation device and the second fixation device, respectively, in the first desired position and the second desired position.

29. The method according to claim 28, further comprising:
attaching the first fixation device to a first articulated element; and
attaching the second fixation device to a second articulated element,
thereby rigidly connecting the first articulated element to the second articulated element.

30. The method according to claim 29, wherein
the first fixation device comprises a cortical bone screw configured to obliquely traverse the lamina and pars interarticularis;
the second fixation device comprises a pedicle screw;
the step of attaching the first fixation device to a first articulated element comprises attaching the cortical bone screw to a cranial vertebral segment, traversing the lamina and ending in the pedicle of the cranial vertebral segment; and
the step of attaching the second fixation device to a second articulated element comprises attaching the pedicle screw to the pedicle of a caudal vertebral segment.
